(19) United States
(12) Patent Application Publication
(10) Pub. No.: US 2026/0018274 A1
(43) Pub. Date: Jan. 15, 2026

(54) System and Method for Efficient Drive-Through and Walk-Through Medical Imaging

(71) Applicant: Morteza Naghavi, Long Beach, CA (US)

(72) Inventor: Morteza Naghavi, Long Beach, CA (US)

(21) Appl. No.: 18/771,542

(22) Filed: Jul. 12, 2024

(51) Int. Cl. G16H 30/20; G06K 7/14; G06Q 40/08; G06V 40/16; G08G 1/00; G16H 15/00

(52) U.S. Cl. CPC G16H 30/20; G06K 7/1404; G06Q 40/08; G06V 40/172; G08G 1/00; G16H 15/00

(57) ABSTRACT

A drive-through medical imaging system and method enables efficient imaging scans for users. The system comprises a facility entrance with signage and teller to guide/communicate with a user's vehicle, a mobile application for the user to answer questions/receive physician order/schedule appointment, a QR code scanner to activate access to an imaging area separated by a gate. The imaging area has a visible scanner, parking area, and operator to guide the user during the scan completed within a set duration. Upon scan completion, the gate opens allowing vehicle exit and entry of another user's vehicle. The system further comprises a facial recognition module for user verification and communication module for transmitting scan results. Additional embodiments feature a walk through version of the system and a standing CT scan booth.

### BACKGROUND

The present invention relates to the field of medical imaging systems and methods. More specifically, the invention pertains to a drive-through and walk-through medical imaging system and method that enables fast and convenient imaging scans for users.

Medical imaging scans, such as computed tomography (CT) scans, are essential diagnostic tools in modern healthcare. However, the process of visiting a physician to obtain a physician order, contacting imaging facilities for scheduling, travelling to the facility, filling out forms and waiting to undergo a scan is highly time-consuming and inconvenient for most people. Traditional imaging facilities require patients to schedule appointments well in advance, fill out lengthy questionnaires, and spend considerable time waiting before the actual scanning procedure.

While this may be needed for sick care where extra care is needed for ill individuals, it is unnecessary and prohibitive for screening purposes where asymptomatic or apparently healthy need to undergo imaging for preventive care. the lengthy and inconvenient process of traditional medical imaging poses significant barriers to adoption for asymptomatic individuals undergoing screening tests for early detection of potentially fatal diseases such as heart disease and lung cancer. These individuals, who are not currently sick, have a much lower tolerance for the extended time commitments required by conventional imaging procedures. As a result, the inconvenience factor becomes a major deterrent to participation in these critical preventive health measures. Furthermore, automation and reducing the need for human resources can reduce the cost therefore making the imaging test more affordable for a larger portion of the population. Currently less than 10% of lung cancer and less than 5% of heart disease are detected via screening which allows for detecting early stage diseases, the rest are diagnosed because of symptoms which are late-stage and often fatal and costly. Similar statistics are true for detection of osteoporosis and fatty liver diseases which are almost always found via symptoms. We have previously patented new AI-enabled technologies for rapid detection of all these pathologies namely heart disease, lung cancer, osteoporosis and fatty liver through a chest CT scan (Ref patent applications or our publications see reference at the bottom). Such an AI-enabled tool helps mass adoption of CT-based screening. However, easy access to CT scanning facilities remains a major barrier to mass adoption and public benefit from such an AI-enabled screening tool. In this invention we aim to eliminate this barrier to mass adoption and as well as financial barriers by using automation technology to reduce costs.

Drive-through services have been widely adopted in various industries to enhance convenience and efficiency for customers. For example, drive-through restaurants, pharmacies, and banks have become increasingly popular. However, the concept of drive-through services has not been extensively explored in the field of medical imaging. Nonetheless, the drive-through COVID-19 testing and vaccination during the COVID pandemic provided a glimpse into the feasibility of such an approach to medical services.

A prior art document, US Patent Application Publication No. 20230398028, discloses a drive-thru medical facility that includes a vehicle pathway and a clinic building. The vehicle pathway comprises an entrance, at least one diagnosis bay for receiving vehicles, an intermediate section, a first exit, and at least one parking space. The clinic building is positioned to be accessible by passengers of vehicles parked in the parking spaces. While this prior art document describes a drive-through medical facility, it does not specifically address the challenges associated with performing medical imaging scans in a drive-through setting. For example, the drive-through system described in this invention is enabled via mobile apps and cloud connectivity unlike U.S. patent application No. 20230398028, this invention does not have cash registration desk or physical spaces for medical staff interactions with patients to gather information or to obtain informed consent, all of which are done via an interactive mobile app.

While drive-through services have been implemented in various industries, and some prior art documents describe drive-through medical facilities, there remains a need for a convenient and efficient system and method specifically designed for performing medical imaging scans in a drive-through setting. The present invention addresses this need by providing a novel drive-through medical imaging system and method that streamlines the entire process, from scheduling and preparation to the actual scanning procedure, thereby improving the overall patient experience, operational efficiency and convenience of use of imaging facilities.

Similar rationales are applicable to a Walk-Through imaging system particularly when the Walk-Through system is powered by AI-enabled automation and is linked to the user's mobile app that manages the entire data gathering, communications and transactions all of which are currently done inefficiently through an arduous and time-consuming processes in hospitals and imaging facilities.

The main concern in such a futuristic Drive-Through and Walk-Through imaging facilities would be when the imaging device such as a CT scan requires X-ray which is known as being hazardous. However, with the development of new X-ray tubes that made super low-dose CT scan imaging possible, and the fact that currently human operators only choose from exiting menu of protocols created in the CT scanner and do not decide on the X-ray dose levels rather than simply click on a drop-down button and choose form the available protocols for example a protocol for coronary artery calcium scan or for lung cancer screening scan. This operation can easily be automated by AI for specialized or solo-function dedicated CT imaging in a drive-through or walk-through CT system that is focused on cardiac and lung cancer screening. It would require a lot of programming to make it a general purpose fully AI controlled imaging versus a highly focused system for high throughput chest CT scanning (lung cancer and heart disease screening) as described here as the main embodiment of the invention. Therefore, this invention although not yet reduced to practice is feasible and addressed a main public health issue (namely missed early detection opportunities for prevention of number 1 and 2 killers i.e. heart disease and lung cancer) due to barrier to access CT scan imaging screening system.

### SUMMARY

The present invention provides a drive-through and walk-through medical imaging system that enables efficient and convenient imaging scans for users. The system comprises a facility with guided entrance and exit, a mobile application for qualifying users, scheduling appointments and generating physician orders, a scanner area with a medical imaging device, and for drive-through a parking area for users' vehicles. The present invention includes embedding visible light cameras in the scanner area and the scanner itself to enable AI-guided management of scanning without human interventions and to generate combined 3D images resulting from visible light of the body's exterior and X-ray imaging of the body's interior enabling co-registration for a more realistic images used for patient education and interactions including augmented reality tools.

In a preferred embodiment, the user drives to the facility's entrance, which may include signs and a teller that is either human or an automated AI. The user must have the mobile application downloaded, questions answered, physician order generated, and scan appointment scheduled. A QR code is provided that activates a green sign to guide the user to move forward to the scanner area.

The user is guided to drive forward inside the facility where the medical imaging device, such as a full-body weight-bearing CT enabling a standing scan position, is located inside a glass wall, allowing visibility. The user exits the vehicle, and an operator guides them into the scanner. The parking area for the vehicle may not be air-conditioned but is separated by a gate that opens to allow the user to drive in and closes afterwards. Once the scan, which typically takes about 2-3 minutes, is completed, the user is instructed to get off the table, walk to their vehicle, and wait for the green light. The gate then opens, allowing the user to leave, and the next vehicle behind them drives in. To reduce scanner vacancy time the entrance can be accessed from both sides of the CT scan room to allow for entry while a patient is leaving to their car the other can be guided to the CT machine.

In an additional embodiment, the method further includes providing a walk-through imaging area at the facility for users without vehicles. Walk-through users entering the facility on foot are guided to the imaging scanner via a dedicated entrance and pathway using directional signage and audio instructions. After performing the imaging scan on the walk-through user, they are directed to exit the imaging area through a separate exit pathway and doorway. The next walk-through user is allowed to enter the imaging area only after the previous walk-through user has exited, ensuring a smooth flow of foot traffic.

By streamlining the entire process, from patient recruitment, scheduling and preparation to the actual scanning procedure, the present invention significantly improves the overall patient experience and operational efficiency of imaging facilities compared to traditional methods.

Additional features and advantages of the invention will be set forth in the description which follows, and in part will be obvious from the description, or may be learned by the practice of the invention. These and other features of the present invention will become more fully apparent from the following description, or may be learned by the practice of the invention as set forth hereinafter.

### BRIEF DESCRIPTION OF THE DRAWINGS

The various exemplary embodiments of the present invention. which will become more apparent as the description proceeds, are described in the following detailed description in conjunction with the accompanying drawings, in which:

FIG. 1 illustrates a system overview diagram of a drive-through medical imaging system.

FIG. 2 depicts a layout of the imaging area.

FIG. 3 details the user flow with the associated mobile application and the mobile application's role in facilitating the CT Scan.

FIG. 4 depicts an embodiment of the CT scan booth wherein the CT scan booth is designed to enable standing scans for the user.

FIG. 5 illustrates an embodiment of the drive-through medical imaging system that reduces scanner vacancy time.

FIG. 6 illustrates a mobile application interface according to an embodiment of the present invention.

### DETAILED DESCRIPTION

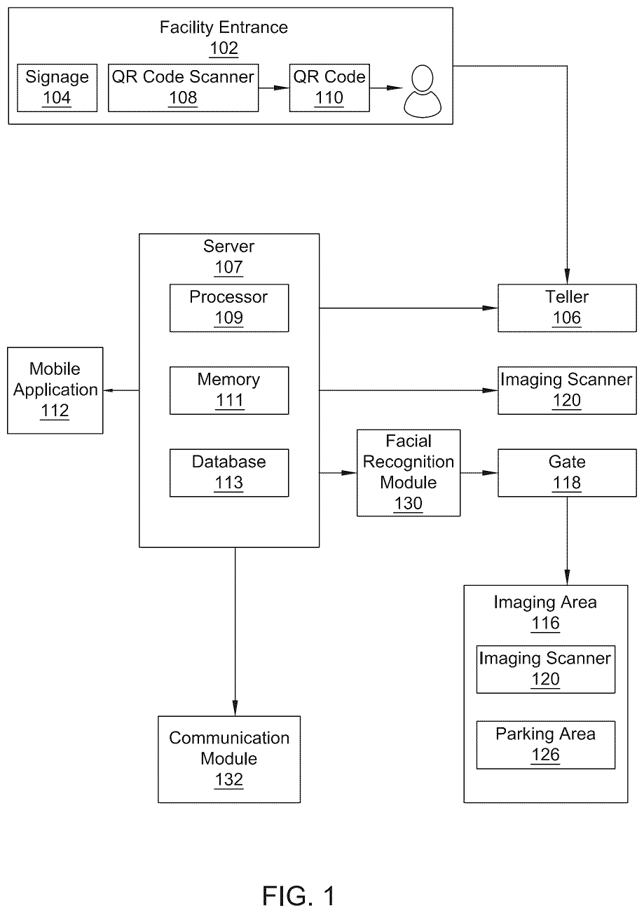

FIG. 1 illustrates a system overview diagram of a drive-through medical imaging system. The system comprises a facility entrance (102) equipped with signage (104) configured to guide a user in a vehicle, and a teller (106) for communicating with the user. In one embodiment, the teller (106) may be a human operator. In another embodiment, the teller (106) may be an automated artificial intelligence (AI) system that employs speech recognition and natural language processing techniques to interact with the user. The system also includes a server (107) equipped with a processor (109), memory (111), and a database (113) for storing and processing data related to the drive-through medical imaging system.

The automated AI system implemented as the teller (106) utilizes advanced speech recognition algorithms, such as deep learning-based models like convolutional neural networks (CNNs) and recurrent neural networks (RNNs). These models are trained on large datasets of speech samples, including various accents, dialects, and noise conditions, to achieve high accuracy and robustness. For example, a CNN architecture with multiple convolutional and pooling layers, followed by fully connected layers, can be employed for feature extraction and classification. The trained models are stored in the memory (111) and executed by the processor (109) of the server (107) to enable real-time speech recognition and processing.

The AI system also employs natural language processing (NLP) techniques, such as named entity recognition (NER), part-of-speech (POS) tagging, and dependency parsing, to analyze and understand the meaning and intent behind the user's spoken words. These techniques enable the system to extract relevant information and provide accurate responses. The NLP module is built using state-of-the-art deep learning architectures, such as the Transformer model, which has been pre-trained on large text corpora and fine-tuned for the specific domain of medical imaging inquiries. The NLP models and associated data are stored in the database (113) and accessed by the processor (109) for real-time natural language understanding and generation. The system further includes a QR code scanner (108) disposed at the entrance (102), adapted to read a QR code (110) provided to the user.

Additionally, the system comprises a mobile application (112), configured to allow the user to answer questions, receive a physician order, schedule an imaging scan appointment, and access instructions for preparing for the scan. The mobile application (112) is developed using modern healthcare informatics software development frameworks, such as FHIR (Fast Healthcare Interoperability Resources), and is available for both iOS and Android operating systems. It integrates with the overall drive-through medical imaging system to enhance user experience and streamline the process, serving as a central hub for users to input necessary information, receive personalized guidance, and manage their appointments. The application (112) exchanges data with the system's backend server (107) using secure communication protocols, such as HTTPS and SSL/TLS, and robust encryption mechanisms to protect sensitive patient information. The server (107) stores and manages the data exchanged with the mobile application (112) in its database (113).

Upon the QR code (110) being read by the scanner (108), a green sign (114) is activated to guide the user to proceed to an imaging area (116) located inside the facility. The imaging area (116) is separated from the entrance (102) by a gate (118). The gate (118) is configured to open to allow the user's vehicle to enter the imaging area (116) and to close during the imaging scan. The server (107) controls the operation of the gate (118) based on the QR code (110) information and the status of the imaging process.

The imaging area (116) includes an imaging scanner (120), such as a CT scanner, that is visible to the user through a glass wall (124). The CT scanner may employ advanced X-ray imaging technology, such as multi-slice CT or dual-energy CT, to produce high-resolution, detailed images of the patient's anatomy. The scanner (120) may also incorporate dose reduction techniques, such as iterative reconstruction algorithms or adaptive dose modulation, to minimize radiation exposure to the patient. The scanner (120) is connected to the server (107), which controls its operation and receives the acquired imaging data for further processing and storage in the database (113).

The area (116) also comprises a parking area (126) adapted to accommodate the user's vehicle and an operator (128) who is configured to guide the user from their vehicle to the scanner (120). The parking area (126) may include sensors, such as ultrasonic sensors or cameras, to assist the user in properly positioning their vehicle and ensuring a safe and efficient parking process. The sensor data is transmitted to the server (107) for analysis and guidance generation. After the imaging scan is completed within a pre-determined duration, the user is signaled to return to their vehicle, and the gate (118) opens upon a green light signal controlled by the server (107) to allow the vehicle to exit. The system is configured to allow entry of another user's vehicle after the previous user's vehicle has exited, ensuring a smooth and continuous flow of patients through the drive-through imaging process.

The system includes additional key components, such as a facial recognition module (130) for verifying the user's identity before allowing vehicle entry. The facial recognition module (130) may utilize advanced computer vision algorithms and deep learning techniques to accurately match the user's face with their pre-registered facial data, providing an additional layer of security and ensuring that only authorized individuals can access the imaging services. The facial recognition models and associated data are stored in the database (113) and executed by the processor (109) for real-time identity verification.

Furthermore, the system includes a communication module (132) for electronically transmitting the imaging scan results to a physician upon completion. The communication module (132) may utilize secure healthcare data exchange standards, such as HL7 or DICOM, to ensure the integrity and confidentiality of the transmitted imaging data. The module (132) may also incorporate encryption and authentication mechanisms to prevent unauthorized access to the sensitive medical information. The server (107) manages the operation of the communication module (132) and facilitates the secure transmission of imaging data from the database (113) to the designated physician.

In one embodiment, the signage (104) at the facility entrance (102) may comprise an interactive display adapted to answer user questions. The interactive display may utilize touchscreen technology and natural language processing to provide users with relevant information, such as directions, wait times, or frequently asked questions about the imaging process. This feature enhances the user experience and reduces the workload on human operators. The interactive display is connected to the server (107), which processes the user queries and generates appropriate responses using the NLP models and data stored in the database (113).

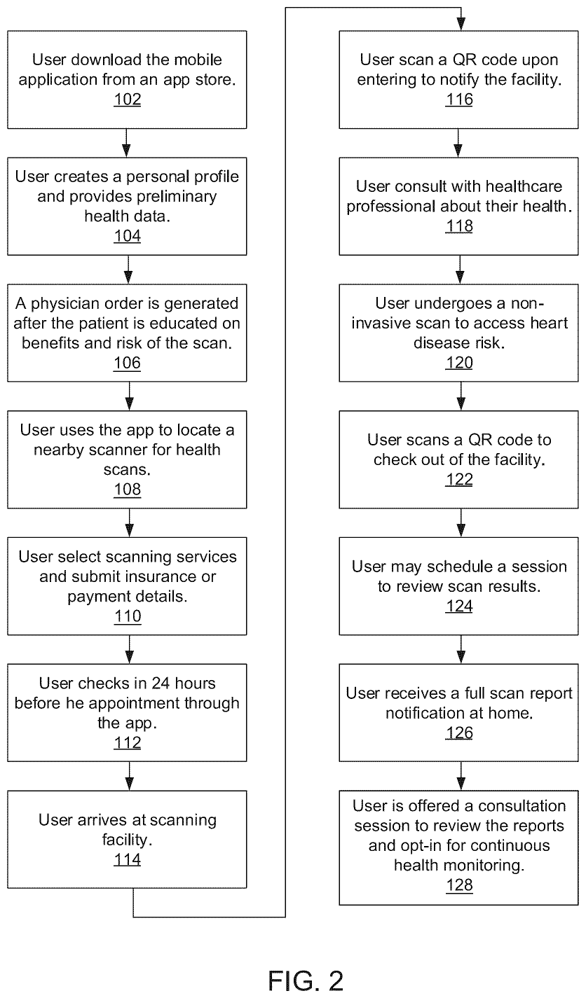

FIG. 2 details the user flow with the associated mobile application (112) and the mobile application's (112) role in facilitating the CT scan, according to an embodiment of the present invention. The method begins with the user downloading the mobile application (“app”) (112) from an app store at step 102. In one embodiment, the user may access the app's (112) download page directly by scanning a quick response (QR) code (110) displayed on a television, monitor, or other QR code display.

Upon launching the app (112) for the first time, the user creates a personal profile at step 104. During the profile creation process, the app (112) collects preliminary health data by presenting the user with a series of questions related to cardiovascular and lung cancer risk factors. The user's responses to these questions enable the app (112) to tailor the subsequent scanning process to the user's specific health needs. In one embodiment, the app (112) may utilize a natural language processing (NLP) engine, such as Google's Dialogflow, to interpret the user's responses and extract relevant health information.

At step 106, the user employs the app's (112) built-in scanner locator feature to find a nearby affiliated scanner (120) capable of performing the desired health scans. Once a suitable scanner (120) has been identified, the user may proceed to schedule an appointment through the app's (112) integrated scheduling interface.

The user selects the specific scanning services required and provides their insurance details at step 108. In the event that the user does not have insurance coverage, the app (112) presents an alternate path at step 110, prompting the user to enter their credit card information to pay for the selected services. The app (112) may employ a secure payment gateway, such as Stripe or PayPal, to process the payment transaction.

At step 112, the user is required to check-in via the app (112) 24 hours prior to their scheduled appointment. This check-in process serves to confirm the user's appointment and allows the scanning facility to prepare for the user's visit, thereby improving the overall efficiency of the service.

On the day of the scheduled scan, the user arrives at the designated facility at step 114. Upon entering the facility, the user scans a QR code (110) at step 116, which automatically checks the user in and notifies the facility's staff to prepare for the user's scheduled service. The app (112) may leverage the device's built-in camera and a QR code scanning library, such as AVFoundation, to facilitate this process.

At step 118, the user engages in a consultation with a healthcare professional. During this consultation, the user may discuss their health concerns, review their medical history, and receive any necessary preparations for the upcoming scan based on the information provided in their app (112) profile. The healthcare professional may access the user's profile information through a secure, HIPAA-compliant web portal that integrates with the app's (112) backend infrastructure.

The user undergoes the Automatic Coronary Artery Calcium (Auto-CAC) scanning process at step 120. This non-invasive scan, which measures the calcium content in the user's arteries as an indicator of heart disease, is designed to be quick and comfortable. The Auto-CAC scan requires no needles, contrast injections, or hospital gowns, and is capable of completing 10 different tests in under 30 seconds. The scan results are automatically uploaded to the user's profile in the app's (112) secure, cloud-based database.

In certain embodiments, the Auto-CAC scan is performed autonomously without necessitating human intervention. User input pertaining to the scan type and any associated parameters is acquired from the patient or the associated healthcare professional via the mobile application (112) executing on the personal computing device, which may comprise a mobile phone or laptop. The user input is securely transmitted from the personal device to the communication module (132) employing healthcare data exchange standards, such as Health Level Seven (HL7) or Digital Imaging and Communications in Medicine (DICOM), to ensure the integrity and confidentiality of the transmitted data. The imaging scanner (120) subsequently utilizes the transmitted data to automatically execute the selected scans based on the received parameters, alleviating the need for onsite programming.

Upon completion of the Auto-CAC scan, the user scans a QR code (110) to check out of the facility at step 122. This check-out process logs the completion of the appointment and may trigger associated billing processes or final administrative tasks.

At step 124, the user has the option to schedule a complimentary review session with a health coach. During this review session, the health coach assists the user in interpreting their scan results and discusses the implications of these results in the context of the user's overall health. The health coach may also provide guidance on how to effectively communicate the scan results to the user's primary care physician.

The user returns home at step 126. Once at home, the user receives a notification from the app (112) indicating that their full scan report and any additional notes or recommendations from the healthcare professional are available for viewing within the app (112). The app (112) may utilize push notification services, such as Apple Push Notification Service (APNs) or Firebase Cloud Messaging (FCM), to deliver these notifications to the user's device.

Finally, at step 128, the user is presented with the option to become a member of an associated community facilitated by the mobile app (112). Membership in this community grants the user access to personalized health plans and ongoing support aimed at preventing serious health issues, such as heart attacks and lung cancer. The app (112) may integrate with a customer relationship management (CRM) platform, such as Salesforce or HubSpot, to manage the user's membership and facilitate ongoing communication and support.

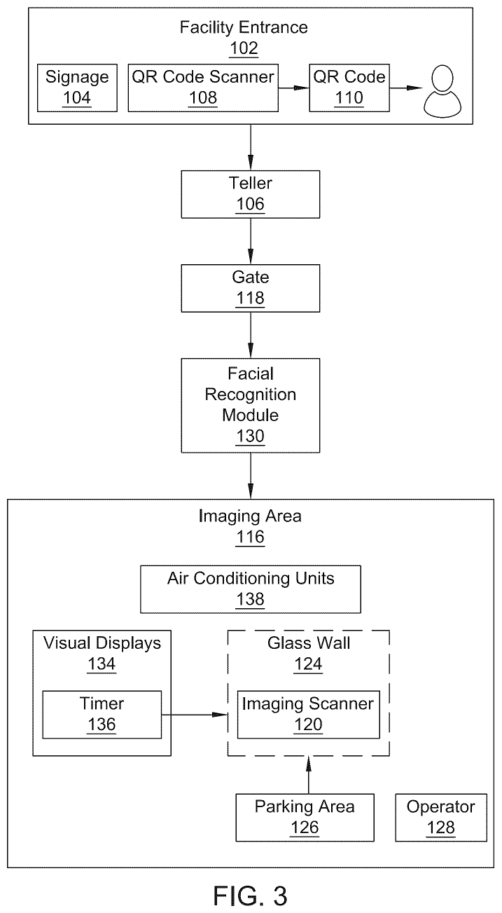

FIG. 3 illustrates an aerial view of the floor plan for a drive-through medical imaging system. The floor plan depicts a facility entrance (102) equipped with signage (104) configured to guide a user in a vehicle, and a teller (106) for communicating with the user. The teller (106) may be a human operator or an automated AI system that employs speech recognition and natural language processing techniques to interact with the user. The system further includes a QR code scanner (108) disposed at the entrance (102), adapted to read a QR code (110) provided to the user.

The floor plan also shows an imaging area (116) located inside the facility, separated from the entrance (102) by a gate (118). The gate (118) is configured to open to allow the user's vehicle to enter the imaging area (116) and to close during the imaging scan. The imaging area (116) includes an imaging scanner (120), such as a CT scanner (122), that is visible to the user through a glass wall (124). Within the imaging area (116), a parking area (126) is provided, adapted to accommodate the user's vehicle. An operator (128) is stationed near the scanner (120) to guide the user from their vehicle to the scanner (120) and back. Visual displays (134) are mounted in clear view, showing a scan duration timer (136) that counts down the time remaining, typically 2-3 minutes. The displays (134) also provide green light signals to indicate when the user should exit the scanner (120) and return to their vehicle.

Arrows on the ground indicate the intended flow and path of the user from the parking area (126) to the scanner (120) and back to their vehicle after the scan is complete. The system is configured to allow entry of another user's vehicle after the previous user's vehicle has exited, ensuring a smooth and continuous flow of patients through the drive-through imaging process.

The floor plan also includes additional key components, such as a facial recognition module (130) for verifying the user's identity before allowing vehicle entry, and air conditioning units (138) for user comfort.

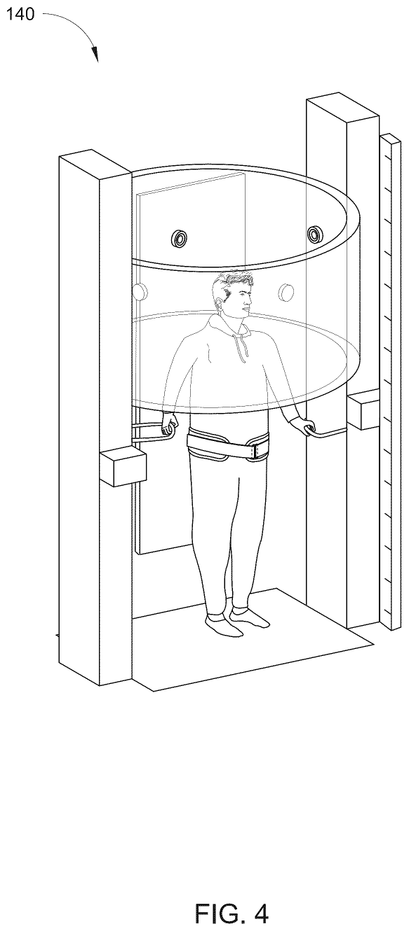

FIG. 4 depicts an embodiment of the CT scan booth (140) wherein the CT scan booth (140) is designed to enable full-body weight-bearing CT scans for the user in a standing position. The booth (140) is an enclosure that allows a user to exit their vehicle and enter for a scan. An external view of the booth (140) shows its compact footprint of approximately 1×1 meter and clearly marked entrance.

The interior of the booth (140) is equipped with sturdy handles (142), positioned for the user to grasp onto to maintain a steady posture during the scan. A visual light signal and audio guide (144) are used to instruct the user on proper positioning and when to exit the booth (140) upon completion of the scan. The system's software enables automatic reconstruction of the 3D volumetric data into multiplanar images for visualization and analysis. The CT scanner includes multiple cameras to view the subject's position accurately and to enable the AI to manage the CT scanning by interacting with the subjects and guiding them to properly position for the CT scan. Also to answer any questions they might have. The AI just like a human radiology tech will be able to answer questions necessary for conducting the CT scan. Additionally the CT scan platform that has an elevator component on the base to elevate the standing subject's chest to center of the CT imaging field of view includes tools to measure the subject's weight and height. The weight is measured using scale electronics inside the base of the elevator platform, and the height is measures using the cameras inside the CT scan and the distance from the step-on elevator platform.

The standing weight-bearing CT scan booth (140) may be deployed at various locations where the drive-through or walk-through medical imaging system is implemented, such as hospitals, clinics, and dedicated imaging facilities. The booth's (140) modular design allows it to be easily installed as an add-on to existing CT or other imaging systems, expanding the range of services offered to include weight-bearing scans for musculoskeletal and other applications. The compact footprint and lower radiation dose compared to conventional CT make it suitable for use in a variety of settings.

In another embodiment of the drive through medical imaging system, a walk-through embodiment is implemented which parallels the drive through system but in a walk-through experience. The system comprises a facility entrance (102) equipped with signage (104) configured to guide a user on foot. An automated AI system employs speech recognition and natural language processing techniques to interact with the user, utilizing advanced algorithms such as CNNs, RNNs, and NLP techniques like named entity recognition NER, POS tagging, and dependency parsing.

The system further includes a QR code scanner (108) disposed at the entrance (102), adapted to read a QR code (110) provided to the user via a mobile application (112). The mobile application (112), developed using modern healthcare informatics software development frameworks like FHIR (Fast Healthcare Interoperability Resources), allows the user to answer questions, receive a physician order, schedule an imaging scan appointment, and access instructions for preparing for the scan.

Upon the QR code (110) being read by the scanner (108), a green sign (114) is activated to guide the user to proceed to a walk-through imaging area (146) located inside the facility. The walk-through imaging area (146) is separated from the entrance (102) by an automated gate (118), configured to open to allow the user to enter the walk-through imaging area (146) and to close during the imaging scan.

The walk-through imaging area (146) includes an advanced imaging scanner (120), such as a multi-slice CT or dual-energy CT scanner, that is visible to the user through a glass wall (124). The scanner (120) employs dose reduction techniques, such as iterative reconstruction algorithms or adaptive dose modulation, to minimize radiation exposure to the patient.

The walk-through imaging area (146) also comprises a waiting area adapted to accommodate the user and an automated guidance system (148) to guide the user from the waiting area to the scanner (120). After the imaging scan is completed within a pre-determined duration, the user is signaled to return to the waiting area, and the gate (118) opens upon a green light signal to allow the user to exit.

The system includes a facial recognition module (130) for verifying the user's identity before allowing entry, utilizing advanced computer vision algorithms and deep learning techniques. A communication module (132) electronically transmits the imaging scan results to a physician upon completion, employing secure healthcare data exchange standards like HL7 or DICOM, along with encryption and authentication mechanisms.

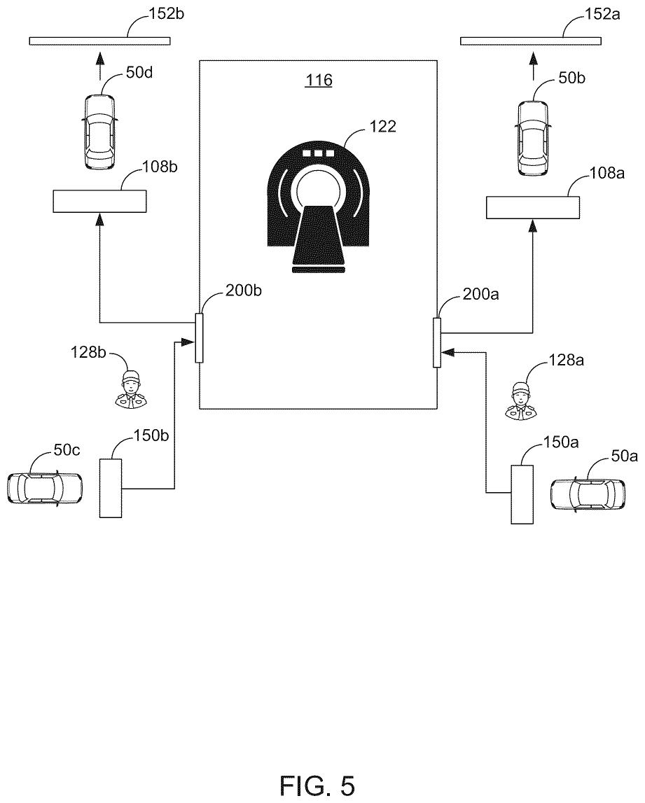

FIG. 5 illustrates an additional embodiment of the drive-through medical imaging system that reduces scanner vacancy time. In this embodiment, the imaging area (116) is accessible from both sides, with separate entrance (150) and exit (152) gates. This configuration allows a first user's vehicle to enter the imaging area (116) via the entrance gate (150) while a second user's vehicle that has completed the imaging scan exits through the exit gate (152). The entrance gate (150) and exit gate (152) operate independently, allowing the next user to be guided by the operator (128) to the CT scanner (122) while the previous user returns to their vehicle and exits the imaging area (116). This simultaneous entry and exit of users' vehicles through the separate gates (150, 152) reduces scanner vacancy time and improves the efficiency of the drive-through medical imaging process. The system's automated control module coordinates the opening and closing of the gates (150, 152) and the guidance provided by the operator (128) to ensure a seamless and continuous flow of users through the imaging area (116).

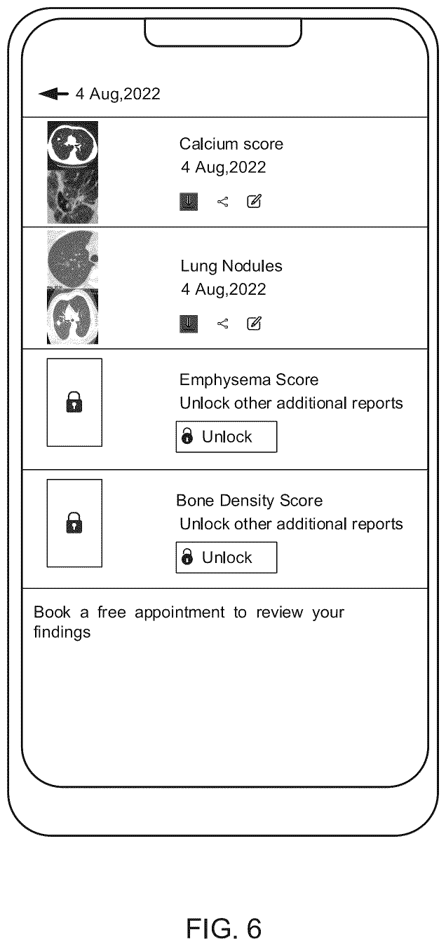

FIG. 6 illustrates a mobile application interface according to an embodiment of the present invention.

The embodiments described herein are given for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

What is claimed is:

1. A system for performing a medical imaging scan in a drive-through system, comprising:
providing a mobile application for user recruitment and qualification, shared-decision and generating a physician order, checking insurance coverage, processing payment, scheduling an imaging scan appointment;
reading, by a QR code scanner at the entrance, a QR code provided to the user;
guiding and communicating with a user in a vehicle at a facility entrance using signage and a teller;
signalling the user to proceed to an imaging area located inside the facility and separated from the entrance by a barrier, upon reading the QR code and verifying the user;
opening the barrier to allow the user's vehicle to enter the imaging area;
closing the barrier during the imaging scan;
guiding, by an operator, the user from the user's vehicle in a parking area to an imaging scanner visible through a glass wall;
performing the imaging scan with built-in scanning protocols according to user's imaging need;
automatically quality checking the images before signaling the user to return to the user's vehicle upon completion of the imaging scan;
opening the barrier to allow the user's vehicle to exit the imaging area upon a QR code reader signal; and
allowing another user's vehicle to enter the imaging area after the previous user's vehicle exits;
transferring the images to AI-enabled image analysis and reporting;
generating AI-enabled reports of the scans;
enabling user to schedule a review of imaging reports with an expert and receiving necessary guidance.

2. The system of claim 1, wherein the imaging scanner is a CT scanner.

3. The system of claim 2, wherein the CT scanner is a standing scanner.

4. The system of claim 1 with embedded cameras with interactive audiovisual systems that allows both human and AI to manage the entire process without any physical touch with the subject.

5. The system of claim 1, wherein the operator is an automated AI system that communicates with the user through video and speech recognition.

6. The system of claim 1, further comprising:
verifying the user's identity using facial recognition before allowing the user's vehicle to enter the imaging area.

7. The system of claim 1, further comprising:
providing a mobile unit that can be transported to different locations, wherein the drive-through or walk-through medical imaging system is set up in the mobile unit.

8. The system of claim . . . wherein the standing CT scan booth comprises one or more handles and back support for the user to hold onto and maintain a steady position during the scan.

9. The system of any one of claims 12-14, wherein the standing CT scan booth is located in one or more of the following: airports, retail stores, and drive-through settings.

10. The system of claim 1, further comprising:
providing a walk-through imaging area at the facility for users without vehicles;
guiding walk-through users entering the facility on foot to the imaging scanner via a dedicated entrance and pathway using directional signage and audio instructions;
performing the imaging scan on the walk-through user;
directing the walk-through user to exit the imaging area through a separate exit pathway and doorway upon completion of the imaging scan; and
allowing the next walk-through user to enter the imaging area after the previous walk-through user has exited.

11. The system of claim 1, wherein the mobile application is further configured to:
provide the user or an associated healthcare professional with a selection of available imaging scan types;
allow the user or the associated healthcare professional to choose one or more desired scan types for the scheduled appointment; and
enable the user or the associated healthcare professional to specify scan parameters associated with the chosen scan types;
wherein the imaging scanner automatically performs the selected scans using the specified parameters upon the user's arrival, without requiring on-site programming.

12. The system of claim 1, wherein the method further comprises:
allowing another user's vehicle to enter the imaging area after the previous user's vehicle exits;
wherein the imaging area is accessible from both sides or same side with separate doors for entrance and exit operating independently to allow simultaneous entry and exit of a first and a second thereby reducing scanner vacancy time and improving efficiency of the drive-through medical imaging process.

13. The system of claim 3 where in the drive-through facility is replaced with a walk-through facility that allows individuals without a vehicle to enter and get scanned.

14. A computerized tomographic scanning system that contains both 3-D X-ray tomographic imaging of the inside of the body and visible light 3-D imaging of the surface of the body wherein the system is able to generate co-registered 3-D X-Ray computed tomography and 3D visible light images of the body.
The system of claim 14 wherein the system is a standing CT scan and contains an elevator platform with built-in weight scale
The system of claim 15 wherein the system has built-in height measurement capability using the distance from the step-on scale and the built-in cameras.
The system of claim 14 where the body scale contains bioimpedance electronics for measuring body composition.
The system of claim 14 where the combined 3D tomographic images of X-ray and visible light imaging is used for augmented reality based education of the patients about their body.
The system of claim 14 where the 3-D X-ray tomographic imaging is replaced with MRI scanner for combined imaging of the exterior surface and the interior organs of the body.
